(19) United States
(12) Patent Application Publication
(10) Pub. No.: US 2009/0281448 A1
(43) Pub. Date: Nov. 12, 2009

(54) Wireless Skin Surface Potential Sensing System and Method

(75) Inventors: Edward J. Wright, Atlanta, GA (US); Phiip R. Kennedy, Duluth, GA (US)

Correspondence Address: BRYAN W. BOCKHOP, ESQ.;BOCKHOP & ASSOCIATES, LLC, 2375 MOSSY BRANCH DR., SNELLVILLE, GA, 30078, US

(73) Assignee: NEURAL SIGNALS, INC., Duluth, GA (US)

(21) Appl. No.: 12/118,687

(22) Filed: May 10, 2008

(51) Int. Cl. A61B 5/04; G09B 21/00

(52) U.S. Cl. 600/546; 340/825.19

(57) ABSTRACT

A sensing apparatus and method allows a patient to control an object by asserting a motor unit action potential. An electromyogram sensor is configured to be placed at a selected location on the patient and to sense a motor unit action potential from the patient. The electromyogram sensor is also configured to generate an EMG signal representative of the motor unit action potential. A personal area network transmitting device that is responsive to the EMG signal is configured to generate a wireless signal that corresponds to the EMG signal. A personal area network receiving device is configured to receive the wireless signal and to convert the wireless signal into an electrical signal. A processor is configured to receive the electrical signal and to generate at least one control signal that has a value corresponding to a state of the electrical signal.

### BACKGROUND OF THE INVENTION

1. Field of the Invention

The present invention relates to electromyogram interfaces and, more specifically, to a wireless electromyogram interface.

2. Description of the Prior Art

Muscle paralysis affects over one hundred thousand people in the United States and approximately one million people worldwide. One class of patients who face severe difficulties in their daily lives is those with locked-in syndrome. Locked-in syndrome patients generally have a cognitively intact brain and a nearly completely paralyzed body. They are alert but cannot move or talk. They face a life-long challenge to communicate. Some patients may use eye movements, blinks or remnants of muscle movements to indicate binary signals, such as “yes” or “no.” One approach used to provide assistance to patients with locked-in syndrome has been described in U.S. Pat. No. 4,852,573, which is hereby incorporated by reference. In this approach, an electrode is implanted into the patient's brain and signals from the electrode may be used to control an electronic device.

To enhance communication with these patients, several devices have been developed to sense skin surface potentials, including electroencephalographic (EEG) potentials and electromyographic (EMG) potentials, to control a computer. These systems can provide patients with the ability to do such tasks as spelling words. Typical EMG control devices receive bioelectrical impulses from EMG sensors attached to the user's body. The EMG sensors sense small electrical impulses generated by motor nerves in various parts of the user's body, such as the forearms and the jaw. One exemplary system for using EMG control is disclosed in U.S. Patent Application Publication No. US-20060004298-A1, which is hereby incorporated by reference.

Unfortunately, most existing systems for EMG control require wires to connect the EMG sensors to the control device (such as a computer). Such wires can make patient care difficult. For example, simply changing the sheets on the patient's bed requires movement of communication wires and can result in their inadvertently being disconnected.

Other systems sense skin surface potentials for other applications. For example electrocardiographic (EKG) skin surface potentials are used in diagnostic procedures. Many systems include wired systems to transmit the sensed potentials to a computer. In certain applications, such wires can be burdensome.

Certain wireless systems for transmitting skin surface potentials wirelessly have been developed. However, certain skin surface potentials (such as EKG and EEG) have extremely low frequencies relative to voice channel systems. Therefore, wireless transmission of such signals requires custom signal transmission and processing technology.

Personal-computer sound cards may be used for analog audio signal acquisition. However, most sound cards are ac-coupled through series capacitors on the signal path. Typical high-pass cutoff frequencies in sound cards tend to be above 20 Hz. This makes it impossible to record waveforms containing dc or low-frequency components, such as those exhibited by skin surface potentials.

Therefore, there is a need for system and method for wireless communication of EMG signals from a patient to a computer.

### SUMMARY OF THE INVENTION

The disadvantages of the prior art are overcome by the present invention which, in one aspect, is a sensing apparatus for allowing a patient to control an object by asserting a motor unit action potential. An electromyogram sensor is configured to be placed at a selected location on the patient and to sense a motor unit action potential from the patient. The electromyogram sensor is also configured to generate an EMG signal representative of the motor unit action potential. A personal area network transmitting device that is responsive to the EMG signal is configured to generate a wireless signal that corresponds to the EMG signal. A personal area network receiving device is configured to receive the wireless signal and to convert the wireless signal into an electrical signal. A processor is configured to receive the electrical signal and to generate at least one control signal that has a value corresponding to a state of the electrical signal.

In another aspect, the invention is a sensing apparatus for allowing a patient to generate a signal by asserting a motor unit action potential. An electromyogram sensor is configured to be placed at a selected location on the patient and to sense a motor unit action potential from the patient. The electromyogram sensor is also configured to generate an EMG signal representative of the motor unit action potential. A personal area network transmitting device that is responsive to the EMG signal is configured to generate a wireless signal that corresponds to the EMG signal.

In yet another aspect, the invention is a method of transmitting control inputs to a device in which an electromyogram action potential is sensed with an electromyogram sensor, thereby generating an EMG signal. The EMG signal is transmitted to a wireless receiver with a wireless personal area network transmitting device. The EMG signal is received with a personal area network receiving device. The EMG signal is processed so as to generate a control signal. A device is controlled based on a state of the control signal.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of an illustrative embodiment of the invention.

FIG. 2 is a schematic diagram showing the embodiment shown in FIG. 1 in use.

FIG. 3 is a schematic diagram showing a second illustrative embodiment.

FIG. 4 is a schematic diagram showing a wireless personal area network embodiment of a skin surface potential transmitting device.

FIG. 5 is a schematic diagram showing a voltage controlled oscillator of the type used in the embodiment shown in FIG. 4.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

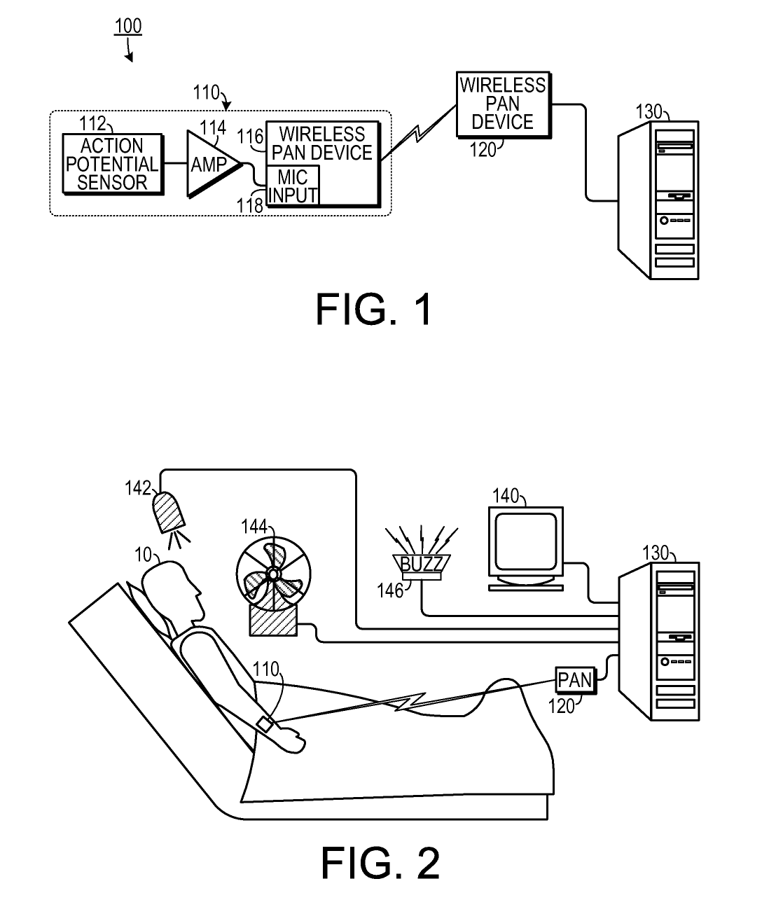

As shown in FIGS. 1 and 2, one embodiment includes a sensing apparatus 100 that allows a patient 10 to control an object by asserting a motor unit action potential. The sensing apparatus 100 includes an electromyogram sensor 110 that is applied to the patient 10 at a location where motor unit action potentials may be sensed. (It should be noted that these locations may vary in patients with locked in syndrome, but they can be located readily by placing an EMG sensing device in several different locations and measuring the output therefrom. Typically, the forearm, wrist and jaw are locations where motor unit action potentials are often sensed.)

The electromyogram sensor 110 typically includes a motor unit action potential sensor 112 (of the type typically know in the art of existing EMG sensing devices). The signal from the motor unit action potential sensor 112 (“the electromyogram signal”) can be amplified by an amplifier 114 and the resulting amplified signal may be sent to the microphone input 118 of a wireless personal area network transmitting device 116. (In one embodiment, the personal area network transmitting device 116 includes a Bluetooth® transmitter. In fact, a commercially available Bluetooth® chip set was used in one embodiment.)

The wireless personal area network transmitting device 116 generates a wireless signal corresponding to the amplified signal, which is received by a wireless personal area network receiving device 120. The wireless personal area network receiving device 120 could include a commercial off the shelf augmented communication system (such as a Bluetooth® system, using the Bluetooth® headset profile). The personal area network receiving device 120 converts the wireless signal into an electrical signal, which is then transmitted to a processor 130, such as a personal computer.

The processor 130 generates control signals corresponding to the electromyogram signal. In a simple embodiment, the control signals act as a computer interface to the patient 10. Feedback may be given to the patient 10 via a computer monitor 140 and the patient may control movement of a cursor on the monitor 140 and may select icons displayed on the monitor 140. The computer may also control one or more external devices, such as, for example: a reading light 142, a fan 144, and an alarm 146. This gives the patient some control over his environment.

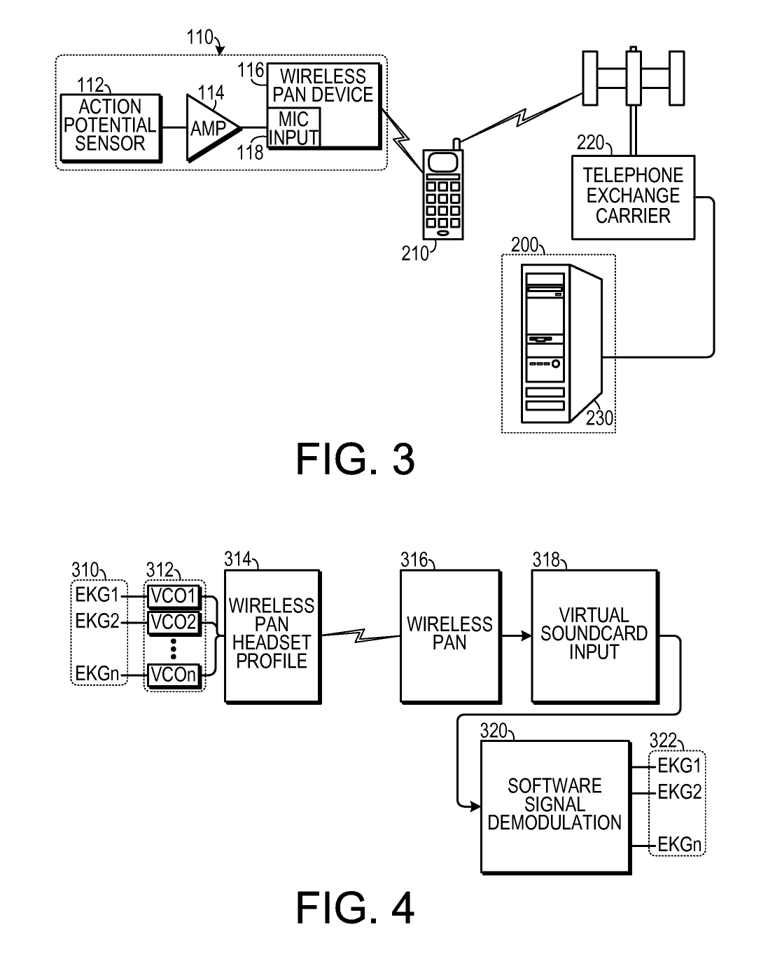

As shown in FIG. 3, one embodiment may be used to relay EMG information to a remote location 200, such as a hospital, lab, service center, or other remote station. In this embodiment, the wireless electromyogram sensor 110 transmits to a Bluetooth®-enabled cellular telephone 210, which transmits the signals to a computer 230 at the remote location 200 via a cellular network 220. The computer 230 could then use the received data for a variety of functions, including analysis, control, notification and the like.

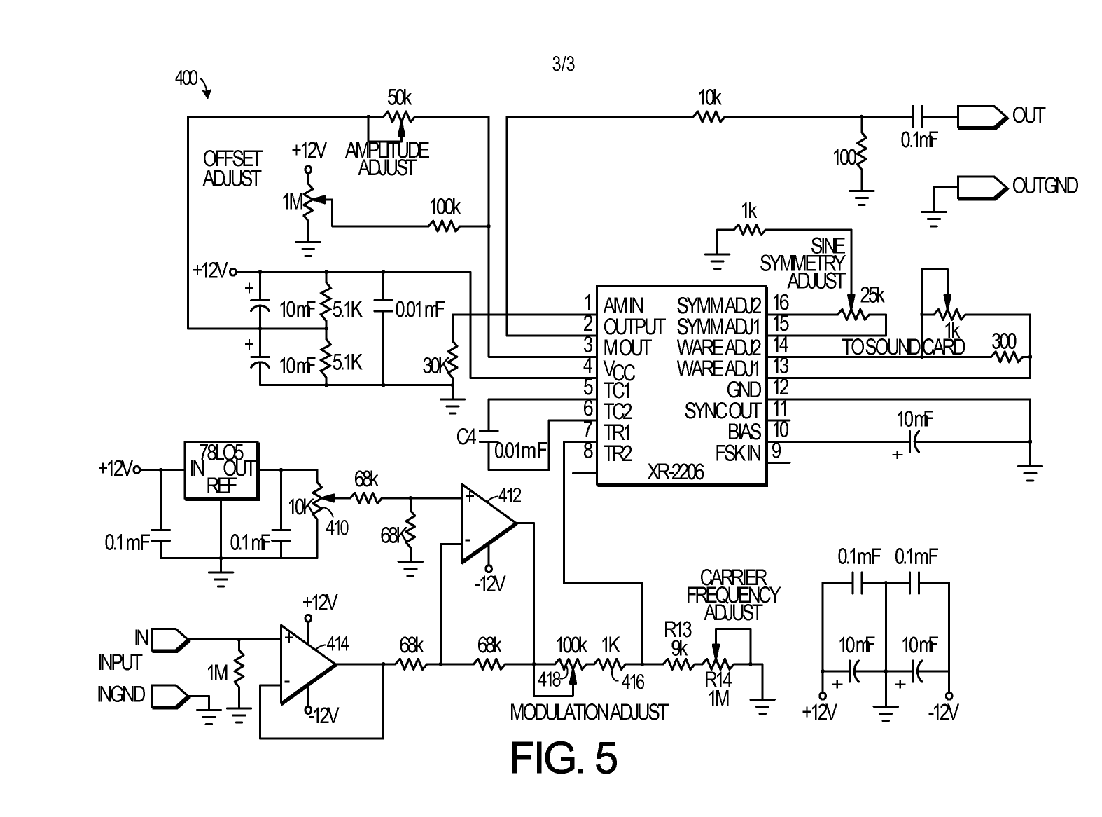

As shown in FIG. 5, a voltage-controlled oscillator (VCO) 400 and some software can turn a sound card into a precision dc-coupled analog-to-digital converter (ADC). The VCO 400 generates an audio tone that varies in frequency as a function of a control signal input. The VCO 400 output is a whistling sound that may be processed by most sound cards. The original signal is recovered by software FM demodulation of the audio signal from the VCO 400. In one embodiment, the integrated circuit labeled XR-2206 is an Exar XR-2206 function generator. The carrier frequency, CF, (in hertz) is: CF=1/(R13+R14)C4.

Using a typical sound card, the carrier frequency may be set in the range of 2 kHz to 10 kHz. The oscillation frequency is modulated by applying a control current in the ±3 mA range to pin 7, which is biased within the XR-2206 at +3 V. Resister 410 sets the offset voltage of op-amp 412 such that zero control voltage applied to op-amp 414 results in zero current across resistor 416 and resistor 418, which adjusts the modulation level (frequency deviation per volt). A good range for most sound cards is around ±80% of the carrier for the full control-voltage input range. The sound card sampling rate should be selected to be at least five times higher than the highest expected VCO frequency.

The following Matlab code generates digitized data (vector x) from the VCO audio (vector y):

Fc = 2144; % Select VCO carrier frequency
vock = 0.176; % Select VCO constant
Fs = 40000; % Select sound card sampling frequency
samptime = 3; % Select sampling time (in seconds)
y = wavrecord(samptime*Fs, Fs, ‘double’); % Sample sound
into y
wavplay(y, Fs); % Play the sound
x=(demod(y,Fc,Fs,’fm’,vcok)); % FM demodulate the sound
cutoff=100/(Fs/2); % Cutoff frequency for post-demodulator
low-pass filter in radians/s is desired cutoff/(½
sampling frequency)
[b,a] = butter(4,cuttof); % Design Butterworth low-pass
filter
xfilt=filter(b,a,x); %Filter demodulated signal

Using a personal area network such as a Bluetooth® headset profile to transmit skin surface potentials has certain advantages, such as: The ability to Amplify and Digitize within millimeters of electrode to skin juncture is made possible because of the size of components of the headset profile. Also, electromagnetic interference (EMI) and motion artifacts cause substantial problems with electric bio-potential signals such as EMG and EEG. The personal area network headset profile significantly reduces EMI and motion artifacts. Such headset profile circuitry is relatively inexpensive and usually includes built-in analog-to-digital capabilities (as well as built-in charging units). The headset profile was also avoids speech latency problems. Some EMG control applications require a response of the EMG switch to be less than 30 ms. Also, a headset profile (via the pairing process) includes built-in encryption and security, which automatically satisfies HIPPA requirements.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A sensing apparatus for allowing a patient to control an object by asserting a motor unit action potential, comprising:
a. an electromyogram sensor configured to be placed at a selected location on the patient and to sense a motor unit action potential from the patient, the electromyogram sensor also configured to generate an EMG signal representative of the motor unit action potential;
b. a personal area network transmitting device, responsive to the EMG signal, that is configured to generate a wireless signal that corresponds to the EMG signal;
c. a personal area network receiving device that is configured to receive the wireless signal and to convert the wireless signal into an electrical signal; and
d. a processor that is configured to receive the electrical signal and to generate at least one control signal that has a value corresponding to a state of the electrical signal.

2. The sensing apparatus of claim 1, further comprising an interface unit, coupled to the processor, that is configured to control an external device based on a current state of the control signal.

3. The sensing apparatus of claim 1, wherein the personal area network transmitting device comprises a Bluetooth® transmitter.

4. The sensing apparatus of claim 1, further comprising an amplifier that is configured to receive the EMG signal from the electromyogram sensor, amplify the EMG signal so as to generate an amplified signal and then transmit the amplified signal to the personal area network transmitting device.

5. The sensing apparatus of claim 4, wherein the amplified signal is coupled to a microphone input of the personal area network transmitting device.

6. The sensing apparatus of claim 1, wherein the personal area network receiving device comprises a Bluetooth® receiver.

7. The sensing apparatus of claim 6, wherein the Bluetooth® receiver comprises a Bluetooth®-enabled cellular telephone.

8. A sensing apparatus for allowing a patient to generate a signal by asserting a motor unit action potential, comprising:
a. an electromyogram sensor configured to be placed at a selected location on the patient and to sense a motor unit action potential from the patient, the electromyogram sensor also configured to generate an EMG signal representative of the motor unit action potential; and
b. a personal area network transmitting device, responsive to the EMG signal, that is configured to generate a wireless signal that corresponds to the EMG signal.

9. A system for transmitting a skin surface potential signals via a wireless voice channel, comprising:
a. a skin surface potential sensor that generates a first signal representative of a skin surface potential;
b. a signal conditioning circuit, responsive to the first signal, that generates a modulated signal representative of the first signal, wherein the modulated signal has a carrier frequency within a bandwidth of the voice channel;
c. a wireless personal area network transmitter, responsive to the modulated signal, that transmits a wireless signal that includes a signal component corresponding to the modulated signal;
d. a wireless personal area network receiver that receives the wireless signal and that generates an electronic signal representative thereof;
e. a demodulation circuit that generates a demodulated signal representative of the electronic signal; and
f. a sound card configured to reconstruct the skin surface potential based on the demodulated signal.

10. A method of transmitting control inputs to a device, comprising the actions of:
a. sensing an electromyogram action potential with an electromyogram sensor, thereby generating an EMG signal;
b. transmitting the EMG signal to a wireless receiver with a wireless personal area network transmitting device;
c. receiving the EMG signal with a personal area network receiving device;
d. processing the EMG signal so as to generate a control signal; and
e. controlling a device based on a state of the control signal.

11. The method of claim 10, wherein the transmitting action comprises the action of employing a Bluetooth® transmitter to transmit the EMG signal.

12. The method of claim 11, wherein the EMG signal is coupled to a microphone input of the transmitter.

13. The method of claim 10, wherein the receiving action comprises the action of employing a Bluetooth® receiver to receive the EMG signal.

14. The method of claim 10, further comprising the action of amplifying the EMG signal before the transmitting step.

15. The method of claim 10, further comprising the action of transmitting the EMG signal to a remote station via a wireless telecommunications network.

16. The method of claim 10, wherein the controlling action comprises the action of controlling a computer user interface.

17. The method of claim 10, wherein the controlling action comprises the action of controlling a lamp.

18. The method of claim 10, wherein the controlling action comprises the action of controlling a fan.

19. The method of claim 10, wherein the controlling action comprises the action of controlling an alarm.
